SEQUENCE LISTING (5 sequences)

SEQ ID NO: 1 - DNA, 597 bp, Artificial Sequence
  Description of Artificial Sequence Synthetic polynucleotide
atgacacctcaaaatattactgatttgtgtgcagaataccacaacacacaaatacatacgctaaatgataagatattttcgtatacagaatctctagctggaaaaagagagatggctatcattacttttaagaatggtgcaacttttcaagtagaagtaccaggtagtcaacatatagattcacaaaaaaaagcaattgaaaggatgaaggataccctgaggattgcatatcttactgaagctaaagtcgaaaagttatgtgtatggaataataaaacgcctcatgcgattgccgcaattagtatggcaaatgggcccgggccccgtcgtaaacgttttgtgaaccaacacctgtgcggctcacacctggtggaagctctctacctagtgtgcggggaacgaggcttcttctacacacccaagacccgccggaaacgtgaggacctgcaggtggggcaggtggagctgggcgggggccctggtgcaggcagcctgcagcccttggccctggaggggtcccgtcgtaagcgtggcattgtggaacaatgctgtaccagcatctgctccctctaccagctggagaactactgcaactaa

SEQ ID NO: 2 - protein, 4 aa, Homo sapiens
GPGP

SEQ ID NO: 3 - protein, 4 aa, Homo sapiens
RRKR

SEQ ID NO: 4 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 5 - protein, 94 aa, Artificial Sequence
  Description of Artificial Sequence Synthetic polypeptide
GPGPRRKRFVNQHLCGSHLVEALYLVCGERGFFYTPKTRRKREDLQVGQVELGGGPGAGSLQPLALEGSRRKRGIVEQCCTSICSLYQLENYCN

(19) United States
(12) Patent Application Publication
(10) Pub. No.: US 2013/0160166 A1
(43) Pub. Date: Jun. 20, 2013

(54) ORAL INSULIN THERAPY

(71) Applicant: University of Central Florida Research Foundation, (US)

(72) Inventor: Henry Daniell, Winter Park, FL (US)

(73) Assignee: University of Central Florida Research Foundation, Inc.

(21) Appl. No.: 13/679,586

(22) Filed: Nov. 16, 2012

Related U.S. Application Data
(60) Provisional application No. 61/561,509, filed on Nov. 18, 2011.

(51) Int. Cl. C07K 14/575; C07K 14/195

(52) U.S. Cl. CPC C07K 14/575; C07K 14/195
USPC 800/305; 530/399; 435/320.1; 800/298; 800/317.3; 435/419

(57) ABSTRACT

This disclosure relates in part to synthesizing a cleavable proinsulin construct in transgenic plants by chloroplast expression.

### CROSS-REFERENCE TO RELATED APPLICATIONS

This application is related to U.S. Provisional Patent Application 61/561,509 filed Nov. 18, 2012, to which priority is claimed under 35 USC 119. The disclosure of this application is incorporated herein by reference.

### STATEMENT OF GOVERNMENT RIGHTS

Development of the following was supported in part by the NIH R01 GM 63879 and USDA 3611-21000-021-02S grants. Accordingly, the U.S. government has certain rights in this invention.

### BACKGROUND

Type 1 diabetes is an autoimmune disorder that results from the T-cells of the body's immune system attacking the 13-cells of the islets of Langerhans in the pancreas. While it represents 5%-10% of all diabetes cases, type 1 diabetes accounts for over 90% of diabetes cases in children. As of 2007, it was reported that diabetes was the seventh leading cause of death in the USA. The main etiology of type 1 diabetes is a drop in insulin levels due to the destruction of the insulin-producing pancreatic 13-cells, which leads to abnormally high blood glucose levels. This can lead to symptoms of the disease such as frequent urination; extreme hunger and thirst; weight loss; fatigue and irritability. However, over time, sustained hyperglycemia leads to a number of other more serious complications such as heart disease, high blood pressure, kidney disease and nervous system disease (CDC, 2008). It is these complications from diabetes that ultimately lead to death if left untreated.

Estimated annual costs in 2007 for diabetes care and other indirect costs for the United States alone totaled $174 billion with roughly $16 billion in excess medical expenditures, including $27 billion going to direct care, $58 billion to treat diabetic complications, and $31 billion in excess general medical costs. It is estimated that approximately $1 out of every $10 health care dollars is attributed to diabetes. As the prevalence of diabetes worldwide continues to increase, these costs are projected to be between $213 and $396 billion by the year 2025. In many developing countries, the annual cost for insulin exceeds 50% of the average annual income. In developed countries, the cost of insulin alone is around $100 per month. Therefore, there is a large need to reduce the economic burden associated with insulin on people dealing with this disease.

### SUMMARY

### Brief Description of the Drawings

FIG. 1: Location of furin cleavage sites. N and C termini are indicated. (a) Native proinsulin. (9 kDa) (b) Proinsulin molecule was modified to include furin cleavage sites and fused to cholera toxin B subunit (CTB). MW of CTB is 11 kDa. MW of modified CTB-proinsulin molecule is 22 kDa. Fusion proteins are expressed by the CTB-Fx3Pris coding region of the pLDutr-CTB-Fx3Pris or pLsLF-CTB-Fx3Pris chloroplast vector.

FIG. 2: Southern analysis of transgenic tobacco containing cholera toxin B subunit (CTB)-Proinsulin with three furin cleavage sites. (a) Map of un-transformed tobacco plastome. Digestion with Afllll yields a 4.2-kb fragment. Broken line represents probe annealing site. (b) Map of transformed tobacco plastome. Digestion with Afllll yields a 6.4-kb fragment. The 5′UTR represents the light-regulated psbA promoter. (c) Southern analysis from T1 generation. A and B represent independent transplastomic lines. WT, untransformed plant.

FIG. 3: Southern analysis of transgenic lettuce containing cholera toxin B subunit (CTB)-proinsulin with three furin cleavage sites. (a) Map of untransformed lettuce plastome. Digestion with Bglll yields a 3.75-kb fragment. Broken line represents probe annealing site. (b) Map of transformed lettuce plastome. Digestion with Bglll yields a 6.3-kb fragment. (c) Southern analysis from T0 generation. 1, 2, 5, 6 and 8 represent independent transplastomic lines. Lowercase letters a and b represent different samples from the same line. WT, untransformed plant.

FIG. 4: Characterization of cholera toxin B subunit (CTB)-proinsulin expressed in tobacco chloroplasts. (a) Coomassie stain. M, marker; S, supernatant; P, pellet; H, homogenate; WT, untransformed plant. S and H: 30 μg per lane. P: 7.5 μg per lane. (b) Western blot probed with anti-CTB antibody showing CTB-PFx3 expression of young, mature and old leaf total protein. 1 μg per lane. CTB: 100 ng CTB standard. Arrows represent the monomer, dimer and trimer bands, respectively. (c) CTB-PFx3 total protein was loaded in varying concentrations and compared to known quantities of CTB standard protein using densitometry. Blot was probed using anti-CTB antibody. (d) Plot of integrated density values (IDV) for quantification of CTB-PFx3 based on standard curve. Broken line shows data points. Solid line: trend line. (e) Percent total protein for young, mature and old leaves based on densitometry values. (f) Quantification of young, mature and old leaves in mg CTB-PFx3 per gram total leaf tissue.

FIG. 5: Characterization of cholera toxin B subunit (CTB)-proinsulin expressed in lettuce chloroplasts. (a) CT8-PFx3 total protein was loaded in varying concentrations and compared to known quantities of CTB standard protein using densitometry. Blot was probed using anti-CTB antibody. (b) Plot of integrated density values (IDV) for quantification of CTB-PFx3 based on standard curve. Broken line shows data points. Solid line: trend line. (c) Percent total protein for young, mature and old leaves based on densitometry values. (d) Quantification of young, mature and old leaves in mg CTBPFx3 per gram total leaf tissue.

FIG. 6: Expression of cholera toxin B subunit (CTB)-proinsulin in dried senescent lettuce leaves. (a) CTB-PFx3 total protein from dried leaf homogenate was loaded in 1 or 2 μg concentrations along with varying dilutions of CTB standard protein. Immunoblot was probed with anti-CTB antibody. (b) CTB-PFx3 total protein from dried leaf homogenate was loaded in 1 or 2 μg concentrations along with varying dilutions of insulin standard protein (shown as multimers). Immunoblot was probed with anti-insulin antibody.

FIG. 7: Furin cleavage assays. Samples were solubilized with 6 M Gu-HCl and 300 mM DTI and subsequently dialysed in 20 mM Na+ PO4-buffer with 500 mM NaCl before addition of Furin protease. (a) Furin digestion of CTB-PFx3. CTB, 100 ng CTB standard protein. −F, CTB-PFx3 before furin cleavage. +F, CTB-PF3 after addition of furin. 1 μg per lane. (b) Silver stained gel after affinity purification of CTB-PFx3. Lane 1, precision plus ladder. Lane 2, Mark 12 low molecular weight ladder. Lane 3, purified CTB-PFx3 1 μg per lane. Lane 4, purified CTB-PFx3 after addition of furin. 1 μg per lane.

FIG. 8: Purification of cholera toxin B subunit (CTB)-proinsulin by affinity chromatography. (a) Coomassie stained gel. DS=dialysed supernatant; FT=flow through; W1=wash 1, W2=wash 2; 2,3=purified fractions; WT=untransformed plant material; CS=control supernatant; CP=control pellet. (b) Coomassie stained gel showing second purification of CTB-PFx3: Lane 1 Marker. Lane 2: control pellet (before purification). Lane 3 Purified CTB-PFx3, Lane 4: Purified CTB-PFx3 after phytate precipitation to remove Rubisco. Lane 5: Control supernatant. All 10 μg loaded. (c) 1 μg of purified CTB-PFx3 samples were loaded using CTB primary antibody. DS=dialysed supernatant: FT=flow through; W1=wash 1, W2=wash 2; 2, 3=purified fractions (d) 1 μg of purified CTB-PFx3 samples were loaded using insulin primary antibody. DS=dialysed supernatant; FT=flow through; W1=wash 1; W2=wash 2; 2, 3=purified fractions. (e) Densitometry shows that after purification, CTB-PFx3 makes up ˜57% of the eluted fractions after the first purification and ˜87% after the second purification as compared to ˜27% of the dialysed solubilized supernatant before passing over the nickel column. (f) The concentration of CTB-PFx3 increases roughly 10-fold (˜320,000 ng/mL) after the first purification and 4.6-fold (140,000 ng/mL) after the second purification over nickel column than the initial concentration of dialysed solubilized supernatant (˜30,000 ng/mL).

FIG. 9: Functional evaluation of cholera toxin B subunit (CTB)-proinsulin in mice delivered by Injection or oral gavage. ANOVA (Dunnett's multiple comparison test) was performed on all groups of mice. (a) Mice were administered two intraperitoneal (IP) injections of PBS only (PBS), purified CTB-PFx3 (Tg) or commercial Insulin (INS Comn.). Blood glucose levels were measured after the first 15 min and each hour (up to three hours) after each injection. Blood glucose levels were statistically lower (**P<0.01) 2 h after the second injection in all groups compared to the PBS only negative control. (b) Mice were administered two oral gavages of untransformed lettuce (WT), tobacco expressing CTB-PFx3 (Tg tobacco), lettuce expressing CTB-PFx3 (Tg lettuce) or two IP injections of commercial Insulin (INS Comm). Blood glucose levels were measured after the first 15 min and each hour (up to three hours) after each injection. Blood glucose levels were statistically lower (**P<0.01; ***P<0.001) 2 h after the second injection in all groups compared to the untransformed (WT) control.

FIG. 10 shows a DNA sequence of 5′-3′ strand of CTB-Proinsulin containing a three furin cleavage sites gene. Start and stop codons are represented in orange. CTB sequence is represented in blue. GPGP hinge is represented in green. Furin cleavage sites are represented in red. Insulin B, C, and A chains are represented in purple reading from left to right, respectively.

### DETAILED DESCRIPTION

The present invention relates to the field of genetic engineering, and in particular to furin cleavable proinsulin constructs expressed by plant chloroplasts, and to methods of making and using the same.

Specifically, insulin is a 51 amino acid peptide hormone with a molecular weight by 5.8 kDa which is derived from its precursor, proinsulin. In normal subjects, the proinsulin is produced in the pancreas a 110 amino acid polypeptide comprising a B-chain, a C-peptide, and an A-chain and is cleaved to produce insulin. For diabetic individuals having an insulin deficiency, commercial preparations of insulin are produced for use mainly in Escherichia coli (E. coli) or yeast as fusion proteins. In E. coli, the A-chain and the B-chain genes are introduced into two separate colonies, and the fusion peptides are produced individually. Then the two chains are mixed together and joined by disulphide bonding. The folded insulin is subsequently purified. Alternatively, insulin is produced from the proinsulin gene in yeast. In known commercial preparations, the C-peptide is removed from the final product.

Certain embodiments relate in part to synthesizing modified proinsulin molecules in transgenic plants. Aspects of the present disclosure provide for chloroplast-derived cleavable proinsulin constructs, for example. In a particular embodiment, tobacco and lettuce chloroplasts were transformed with expression vectors to express a cholera toxin B subunit fused with human proinsulin (A, B, C peptides) containing three furin cleavage sites. Chloroplasts of tobacco and lettuce plants are particularly suitable because of their ability to process eukaryotic proteins, including folding and formation of disulfide bridges, thereby eliminating the need for expensive post-purification processing. Lettuce leaves accumulate proinsulin up to 53% total leaf protein (TLP). Tobacco is further an ideal choice for this purpose because of its large biomass, ease of scale-up (million seeds per plant) and genetic manipulation. The expressed proinsulin from tobacco leaves was purified up to 98% using metal affinity chromatography without any His-tag. Furin protease cleaved the expressed proinsulin constructs in vitro. In addition, delivery of the expressed proinsulin constructs bioencapsulated in plant cells or injectable delivery into mice showed reduction in blood glucose levels similar to processed insulin. Advantageously, in certain embodiments, the expressed cleavable proinsulin constructs comprise the C-peptide for administration to a subject. C-peptide is potentially very useful because it aids in long-term treatment of diabetic complications, including stimulation of nerve and renal functions.

### 1.1 DEFINITIONS

As used herein, the terms “administering” or “administration” of an agent, drug, or peptide to a subject includes any route of introducing or delivering to a subject a composition as described herein to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the term “C-peptide” includes a marker to determine the extent of insulin release in patients. C-peptides aid in long-term treatment of diabetic complications including stimulation of nerve and renal functions. During insulin biosynthesis in the pancreas, the C-peptide is cleaved off via the action of prohormone convertases 2 and 3 at the B-chain/C-peptide and C-peptide/A-chain junctions, respectively, and insulin (51aa) is formed via disulphide bonds between the A- and B-chains.

As used herein, the term “CTB” refers cholera toxin B subunit. Cholera toxin is a protein complex comprising one A subunit and five B subunits. The B subunit is nontoxic and important to the protein complex as it allows the protein to bind to cellular surfaces via the pentasaccharide chain of ganglioside. CTB is useful as a marker or tracer.

As used herein, the term “chloroplast” includes organelles or plastids found in plant cells and other eukaryotic organisms that conduct photosynthesis. Chloroplasts capture light energy to conserve free energy in the form of ATP and reduce NADP to NADPH through a complex set of processes called photosynthesis. Chloroplasts contain chlorophyll. Chloroplasts have a higher copy number and expression levels of the transgene. Each chloroplast may contain up to 100 genomes, while each plant cell may contain up to 100 chloroplasts. Therefore, each plant cell may contain as many as 100000 chloroplast genomes which results in high expression levels of proteins expressed via the chloroplast genome. Chloroplasts further offer gene containment through maternal inheritance as the chloroplast genome is not transferred through pollen unlike nuclear genomic DNA. Chloroplasts have the ability to transcribe polycistronic RNA and can perform the correct processing of eukaryotic proteins including the ability to carry out post-translational modifications such as disulphide bonding, assembly of multimers and lipid modifications.

As used herein, a “composition,” “pharmaceutical composition” or “therapeutic agent” all include a composition comprising a cleavable proinsulin construct as described herein. Optionally, the “composition,” “pharmaceutical composition” or “therapeutic agent” further comprises pharmaceutically acceptable diluents or carriers.

As used herein, the term “diabetes” or “diabetic” or “diabetes mellitus” refer to a physiological disorder that develops when glucose regulation is altered such that a subject is hyperglycemic corresponding to abnormally high levels of circulating glucose, respectively, correlating with altered circulating insulin levels, for examples, due to diminished amounts in circulating insulin, (e.g., lack of release from beta cells, loss of beta cells, etc.), due to resistance to insulin, typically resulting in increased amounts of circulating insulin, (e.g., lack of insulin binding to its receptor, lack of insulin bound receptor mediated enocytosis, lack of glucagon release or lack of glucagon production, etc.), and the like.

As used herein, the terms “type 1 diabetes” or “type 1 diabetes mellitus” or “insulin dependent diabetes” refer to a disorder developing from diminished secretion of insulin, for example, when insulin-producing cells of the pancreas have reduced capability to secrete insulin from malfunction or cell death. In other words, type 1 diabetic subjects are typically hyperglycemic, specifically because they lack insulin-producing cells due to autoimmune destruction.

As used herein, the term “type 2 Diabetes” or “type 2 (II) diabetes mellitus” refers to a disorder developing from diminished function of insulin, for example, when circulating insulin fails to regulate glucose. Type II diabetic patients are hyperglycemic, specifically due to insufficient insulin secretion and/or resistance to its action on target cells to take up glucose from the blood, or both, in the absence of autoimmune destruction of the insulin-producing cells.

As used herein, the terms “disease,” “disorder,” or “complication” refers to any deviation from a normal state in a subject. In preferred embodiments, the methods and compositions of the present disclosure are useful in the diagnosis and treatment of type 1 diabetes.

As used herein, by the term “effective amount,” “amount effective,” “therapeutically effective amount,” or the like, it is meant an amount effective at dosages and for periods of time necessary to achieve the desired result.

As used herein, the term “expression” in the context of a gene or polynucleotide involves the transcription of prokaryotic genes at high level in plant chloroplasts.

As used herein, the term “furin cleavage site” refers to an amino acid sequence that can be recognized by a furin endoproteanase molecule and cleaved, resulting in two or more amino acid fragments.

As used herein, the term “furin protease” includes enzymes that will act and cleave an amino acid sequence at a furin cleavage site.

As used herein, the term “preventing” means causing the clinical symptoms of the disease state not to develop, e.g., inhibiting the onset of disease, in a subject that may be exposed to or predisposed to the disease state, but does not yet experience or display symptoms of the disease state.

As used herein, the terms “proinsulin subunit” or “proinsulin” generally refers to a molecule that is a precursor to insulin.

As used herein, the term “subject” refers to any animal (e.g., a mammal), including, but not limited to, humans, non-human primates, rodents, and the like, which is to be the recipient of a particular treatment.

As used herein, the terms “treating” or “treatment” or “preventing” refers to both therapeutic treatment and prophylactic or preventative measures, wherein the object is to prevent or slow down (lessen) the targeted pathologic condition or disorder.

As used herein, the terms “total leaf protein” (TLP) includes leaf amino acid compositions, enzymes, structural proteins and free protein amino acids.

As used herein, the term “vector” refers to a nucleic acid molecule capable of transporting another nucleic acid to which it has been linked.

In accordance with a first aspect of the present disclosure, there is provided a cleavable proinsulin construct comprising a cholera toxin B (CTB) subunit fused to a proinsulin subunit and at least one furin cleavage site on the cleavable proinsulin construct.

In accordance with another aspect of the present disclosure, there is provided a DNA construct that encodes, upon expression in a plant cell, a cleavable proinsulin construct comprising a CTB subunit fused to a proinsulin subunit and at least one furin cleavage site on the cleavable proinsulin construct.

In accordance with yet another aspect of the present disclosure, there is provided a transgenic plant transformed with a DNA construct that encodes, upon expression in a plant cell, a cleavable proinsulin construct comprising a CTB subunit fused to a proinsulin subunit and at least one furin cleavage site on the cleavable proinsulin construct.

In accordance with yet another aspect of the present disclosure, there is provided a plant cell having chloroplasts transformed to express a cleavable proinsulin construct comprising a CTB subunit fused to a proinsulin subunit and at least one furin cleavage site on the cleavable proinsulin construct.

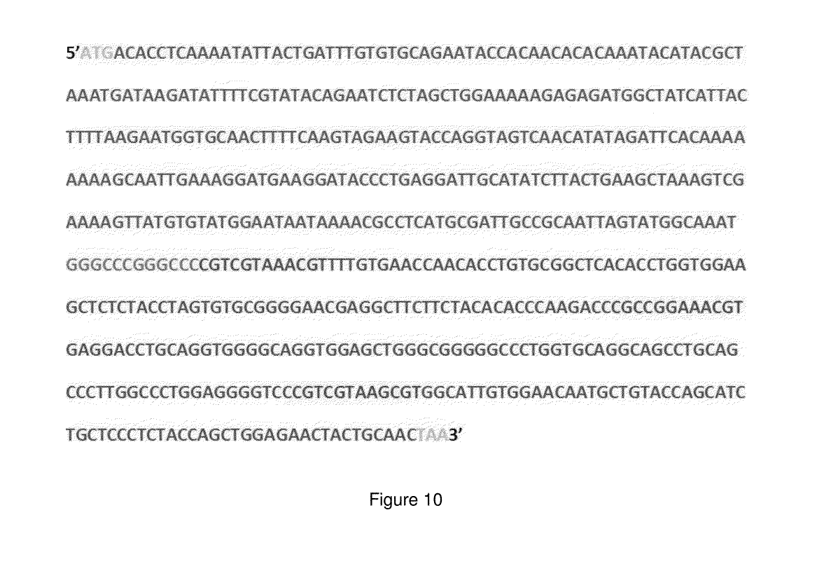

In accordance with yet another aspect of the present disclosure, there is provided a gene sequence for expressing a cleavable proinsulin construct, the gene sequence comprising a sequence according to SEQ. ID. NO: 1 (See FIG. 10).

In accordance with yet another aspect of the present disclosure, there is provided a gene sequence for expressing a cleavable proinsulin construct comprising a CTB subunit fused to a proinsulin subunit and at least one furin cleavage site on the cleavable proinsulin construct.

In accordance with yet another aspect of the present disclosure, there is provided a transformed chloroplast genome that has been transformed with a vector comprising a heterologous gene that expresses a cleavable proinsulin construct as described herein.

In accordance with yet another aspect of the present disclosure, there is provided a method for treating or preventing type 1 diabetes comprising administering to a subject a composition comprising a cleavable proinsulin construct comprising a cholera toxin B (CTB) subunit fused to a proinsulin subunit and at least one furin cleavage site on the cleavable proinsulin construct. In one embodiment, the composition further comprises a pharmaceutically acceptable carrier.

In accordance with yet another aspect of the present disclosure, there is provided a method for treating or preventing hyperglycemia comprising administering to a subject a composition comprising a cleavable proinsulin construct comprising a cholera toxin B (CTB) subunit fused to a proinsulin subunit and at least one furin cleavage site on the cleavable proinsulin construct.

In one embodiment, in any of the embodiments of the constructs, compositions, and/or methods described herein, the proinsulin subunit comprises a C peptide, an A chain, and a B chain and three furin cleavage sites. The C peptide is disposed between the A chain and the B chain. First and second ones of the furin cleavage sites are flanked on opposed sides of the C-peptide and a third furin cleavage site is located at a junction between the CTB subunit and the proinsulin subunit.

### 1.1 Proinsulin Construct

Proinsulin is produced in the pancreas as a 110 amino acid polypeptide containing a 24 amino acid signal sequence and the 86 amino acid proinsulin molecule consisting of the B-chain (30aa), C peptide (34 aa) and A-chain (21 aa). During insulin biosynthesis in the pancreas, the C-peptide is cleaved off via the action of prohormone convertases 2 and 3 at the B-chain/C-peptide and C-peptide/A-chain junctions, respectively, and insulin (51 aa) is formed via disulphide bonds between the A- and B-chains (Steiner, 1998; Shaw et al., 2002). An exemplary nucleic acid sequence for wild-type proinsulin is described in Groskreutz et al. J. Biol. Chem., 269:6241-6245, 1994, for example. It is contemplated that the proinsulin chains (A, B, and/or C) may be separated by linker sequence if desired. Further, it is understood that the proinsulin or proinsulin subunit may be any portion of a proinsulin sequence so long as the translated nucleotide sequence can be processed into an insulin molecule that stimulates glucose uptake by a cell.

### 1.2 Furin Cleavage Sites

The cleavable proinsulin constructs may further include at least one furin cleavage site. The furin cleavage site may be any amino acid sequence that can be recognized by a furin molecule and cleaved, resulting in two or more amino acid fragments. Furin is an endoprotease present in the constitutive secretory pathway and cell surface of virtually all cells (Taylor et al., 2003). The consensus sequence for furin cleavage is the C-terminal arginine in the amino acid sequence Arg-X-Lys/Arg-Arg (Thomas, 2002). The introduction of furin consensus sequences at the B-chain/C-peptide and the C-peptide/A-chain of the proinsulin molecule has been demonstrated to increase the processing of proinsulin to mature insulin in a wide variety of non-neuroendocrine cells, including fibroblasts, myoblasts, epithelial cells and lymphocytes (Groskreutz et al., 1994; Fujimoto et al., 2005; Tatake et al., 2007). In contrast, native proinsulin is processed in the pancreatic β-cells via prohormone convertases PC2/PC3. Non-neuroendocrine cells therefore do not possess the ability to process proinsulin into mature insulin. This processing is necessary for function of insulin, so it has been discovered that addition of furin cleavage sites is necessary to facilitate processing in the gut. Although several different furin cleavage sites have been engineered, there seems to be the best processing (87%) of human proinsulin to unmodified mature human insulin using the Arginine Arginine Lysine Arginine (RRKR) furin cleavage sequence in adult fibroblasts (Shaw et al., 2002). Therefore, in one embodiment, this sequence was engineered into proinsulin in plants to form a cleavable proinsulin molecule as claimed.

### 1.3 C-Peptide

Advantageously, aspects of the present disclosure provide a cleavable protein construct is configured to deliver a C-peptide of a proinsulin subunit or proinsulin to the subject when administered to the subject. C-peptide is currently lacking in commercially available insulin. This is of great importance because type 1 diabetic patients fail to produce C-peptide, an essential molecule that once was thought to be biologically inactive. C-peptide is located in between the B-chain and A-chain of the proinsulin molecule. Only recently, C-peptide has been shown to function independently of insulin. C-peptide acts via G-protein activation, which signals the cell to open Ca²⁺ channels allowing for an influx of cellular Ca²⁺. This leads to stimulation of eNOS, Na⁺, K⁺-ATPase and the MAP kinase pathway (Wahren, 2004). This action of C-peptide is thought to be the mechanism behind the beneficial effects seen upon restoration of C-peptide levels in type I diabetic animals and humans (Johansson et al., 2000; Sima and Li, 2005; Hills and Brunskill, 2009). Stimulation of the activities of Na⁺, K⁺-ATPase and endothelial nitric oxide synthase (eNOS) is essential for nerve function (Ekberg et al., 2003; Wahren et al., 2007), and the stimulation of Na⁺, K⁺-ATPase also leads to correction of renal structure and function (Rebsomen et al., 2008).

### 1.4 Chloroplasts Transformation

Chloroplast genetic engineering in plants offers several advantages over nuclear transformation. Some of these advantages include higher copy number and expression levels of the transgene. Each chloroplast may contain up to 100 genomes, while each plant cell may contain up to 100 chloroplasts. Therefore, each plant cell may contain as many as 10 000 chloroplast genomes which results in high expression levels of proteins expressed via the chloroplast genome. Expression levels of up to 70% total soluble protein (Oey et al., 2009) and 72% total leaf protein (Ruhlman et al., 2010) of biopharmaceutical proteins have been reported in transgenic chloroplasts, which are the highest levels reported in published literature in this field. Chloroplasts offer gene containment through maternal inheritance, as the chloroplast genome is not transferred through pollen unlike nuclear genomic DNA (Daniell, 2007).

Tobacco is ideal because a tobacco plant produces a million seeds, thereby facilitating large-scale production. Tobacco provides relative ease of genetic manipulation. Old tobacco leaves accumulated proinsulin up to 47% of total leaf protein (TLP). Based on the yield of only monomers and dimers of proinsulin (3 mg/g leaf, a significant underestimation), with a 50% loss of protein during the purification process, one acre of tobacco could yield up to 20 million daily doses of insulin per year. Lettuce leaves are ideal because they were shown to accumulate proinsulin up to 53% TLP. Accumulation can be so stable that up to 40% proinsulin in TLP was observed even in senescent and dried lettuce leaves, facilitating their processing and storage in the field.

### 1.5 Variants

Specific polypeptide sequences refer to the full length amino acid sequences or biologically active variants thereof. Preferably, naturally or non-naturally occurring polypeptide variants have amino acid sequences which are at least about 55, 60, 65, or 70, preferably about 75, 80, 85, 90, 96, 96, or 98% identical to the full-length amino acid sequence or a fragment thereof. Percent identity between a putative polypeptide variant and a full length amino acid sequence is determined using the Blast2 alignment program (Blosum62, Expect 10, standard genetic codes). Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of polypeptide can be found using computer programs well known in the art, such as DNASTAR software. Whether an amino acid change results in a biologically active LecA polypeptide can readily be determined by assaying for native activity, as described for example, in the specific

Reference to genetic sequences herein refers to single- or double-stranded nucleic acid sequences and comprises a coding sequence or the complement of a coding sequence for polypeptide of interest. Degenerate nucleic acid sequences encoding polypeptides, as well as homologous nucleotide sequences which are at least about 50, 55, 60, 65, 60, preferably about 75, 90, 96, or 98% identical to the cDNA may be used in accordance with the teachings herein. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of nucleic acid sequences which encode biologically active polypeptides also are useful polynucleotides.

Variants and homologs of the nucleic acid sequences described above also are useful nucleic acid sequences. Typically, homologous polynucleotide sequences can be identified by hybridization of candidate polynucleotides to known polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% basepair mismatches.

Species homologs of polynucleotides referred to herein also can be identified by making suitable probes or primers and screening cDNA expression libraries. It is well known that the Tm of a double-stranded DNA decreases by 1-1.5 OC with every 1% decrease in homology (Bonner et al., J. Mol. Biol. 81, 123 (1973). Nucleotide sequences which hybridize to polynucleotides of interest, or their complements following stringent hybridization and/or wash conditions also are also useful polynucleotides. Stringent wash conditions are well known and understood in the art and are disclosed, for example, in Sambrook et al., MOLECULAR CLONING: A LABORATORY MANUAL, 2nd ed., 1989, at pages 9.50-9.51.

### 1.6 Chloroplast Vectors

Methods, vectors, and compositions for transforming plants and plant cells are taught for example in WO 01/72959; WO 03/057834; and WO 04/005467. WO 01/64023 discusses use of marker free gene constructs. Any vector capable of plastid transformation, particularly of chloroplast transformation, may be utilized in aspects of the present invention. Such vectors include chloroplast expression vectors such as pLDutr, pLsLF, pUC, pBR322, pBLUESCRIPT, pGEM, and others identified by Daniell in U.S. Pat. No. 5,693,507 and U.S. Pat. No. 5,932,479. In some embodiments, the vectors may include flanking sequences that are located outside of the inverted repeat of the chloroplast genome. As an illustrative embodiment for the vectors, the inventors inserted the subject gene constructs into a pLD vector as previously established by the Daniell laboratory (Verma and Daniell, 2007; Verma et al., 2008) or the pLsLF vector (Ruhlman et al., 2007, 2010) under the control of the light-regulated psbA region located within the 5′ UTR. Both vectors contain the aadA gene that confers resistance to spectinomycin and are driven by the upstream prrn promoter. The chloroplast vectors may also include native tobacco or lettuce DNA flanking regions (trnI/trnA) in order to facilitate homologous recombination.

### 1.7 Bombardment

Introduction of the chloroplast vectors into plants for expression of the cleavable proinsulin constructs in the chloroplasts of the respective plants described herein may be accomplished by any suitable method known in the art. In one embodiment, the chloroplast vectors may be introduced in the plants by bombardment techniques as are known in the art. For example, in one embodiment, the chloroplast vectors are introduced by microprojectile bombardment. See e.g., Daniell H. (1997).

### 1.8 Compositions

Compositions comprising a cleavable proinsulin construct as described herein may be in any suitable form for administration to a subject, such as by oral or intravenous administration. For example, a composition comprising a cleavable proinsulin construct may be in the form of tablets, capsules, granules, powders and the like with at least one pharmaceutical carrier. Exemplary pharmaceutically acceptable carriers include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In a particular embodiment, the present disclosure provides a composition bioencapsulated in choroplasts of a plant cell. The chloroplasts have been modified to express the cleavable proinsulin construct. The cleavable proinsulin construct may be produced in the modified chloroplasts and barring rupture or some other disruptive stimulus, the expressed protein is pooled and stored in the chloroplast. Thus the chloroplast acts as a protective encapsulation of the protein sample.

Further details on techniques for formulation and administration can be found in the latest edition of REMINGTON′S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration. In addition, if desired, the compositions may contain minor amounts of auxiliary substances such as wetting or emulsifying agents, pH buffering agents, or adjuvants. Plant, juice, grain, leaves, tubers, stems, seeds, roots or other plant parts of a transgenic plant expressing the cleavable proinsulin construct as herein may be administered to the subject.

### 1.9 Delivery to Subject

The composition may be delivered to the subject via any suitable route of administration such that the composition may perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another. In one embodiment, the composition is delivered intravenously. In another embodiment, the composition is delivered intravenously.

The exact dosage will be determined by the practitioner, in light of factors related to the subject that requires treatment. Dosage and administration may be adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy.

### 1.10 Treatment of Diseases

The methods and compositions as described herein may be utilized in the treatment and/or prevention of any disorder or condition wherein a biological response is shown. In one embodiment, the methods and compositions described herein may be utilized for the treatment of diabetes mellitus, and in a particular embodiment, type 1 diabetes. The etiology of type 1 diabetes is a drop in insulin levels due to the destruction of the insulin-producing pancreatic β-cells, and therefore this leads to abnormally high blood glucose levels. This can lead to symptoms of the disease such as frequent urination, extreme hunger and thirst, weight loss, fatigue and irritability. However, over time, sustained hyperglycemia leads to a number of other more serious complications such as heart disease, high blood pressure, kidney disease and nervous system disease (CDC, 2008). The compositions described herein may be administered to a subject for the treatment or prevention of any of the disease states or symptoms as described herein.

### 2. RESULTS

### 2.1 Chloroplast Vectors for Expression of Cleavable Proinsulin

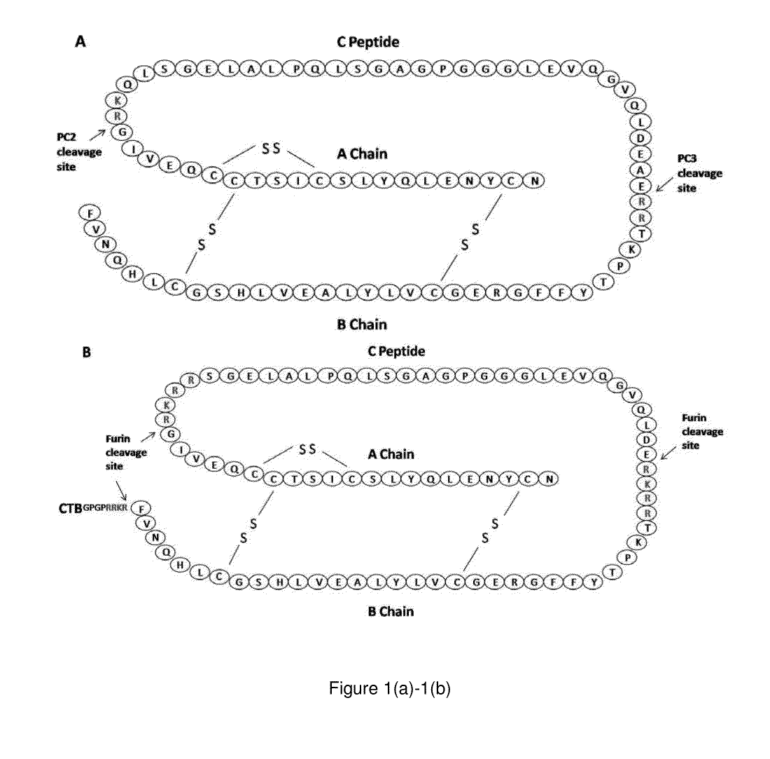

The CTB-PFx3 gene construct was inserted into the pLD vector as previously established by the Daniell laboratory (Verma and Daniell, 2007; Verma et al., 2008) or the pLsLF vector (Ruhlman et al., 2007, 2010) under the control of the light-regulated psbA region located within the 5′ UTR. Both vectors contain the aadA gene that confers resistance to spectinomycin and are driven by the upstream prrn promoter. Chloroplast vectors include native tobacco or lettuce DNA flanking regions (trnI/trnA) in order to facilitate homologous recombination. The CTB gene was separated from the proinsulin gene via a Glycine-Proline-Glycine-Proline (GPGP) hinge to avoid steric hindrance, followed by a furin cleavage site (RRKR) to enable removal of the CTB following delivery. Two additional furin cleavage sites were engineered at the B-chain/C-peptide and the C-peptide/A-chain junctions, replacing the native PC2 and PC3 cleavage sites as previously described (Shaw et al., 2002). The location of engineered cleavage sites and disulphide bonds are compared with native proinsulin in FIG. 1. The location of CTB fusion is also shown.

### 2.2 Chloroplast Transformation and Evaluation of Homoplasmy

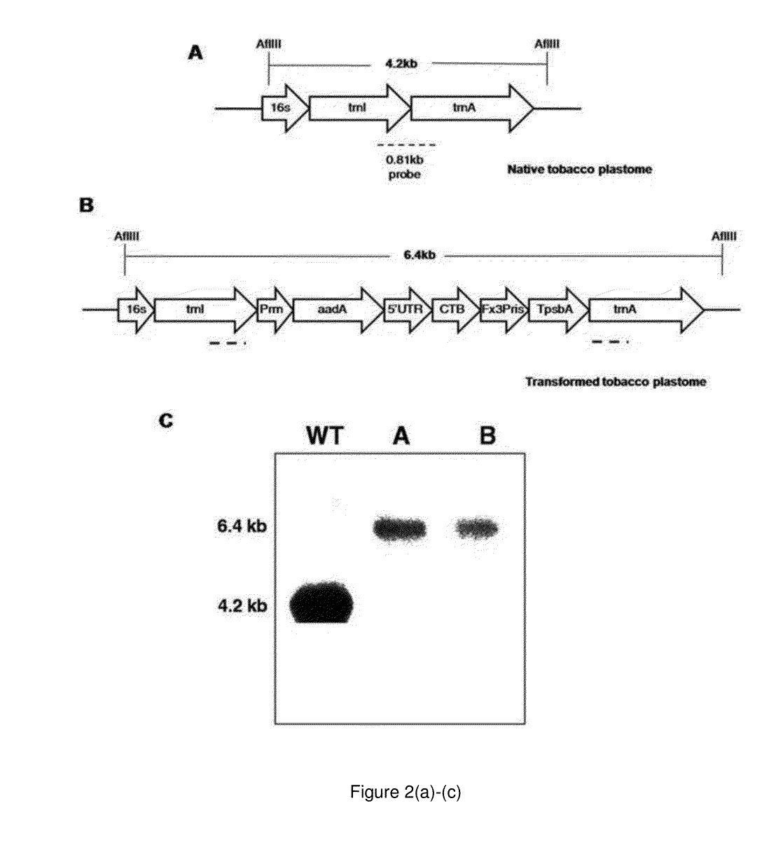

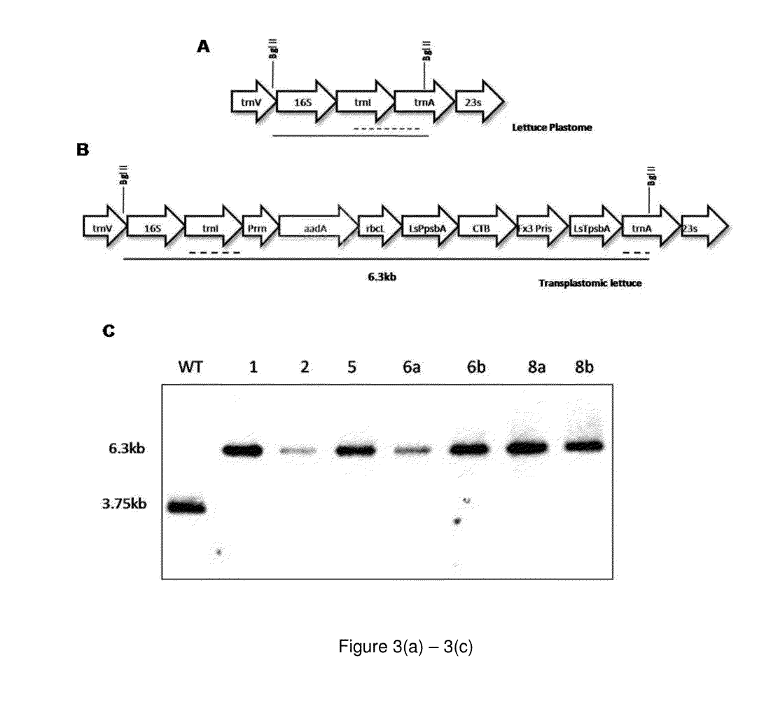

Chloroplast vectors pLDutr-CTB-Fx3Pris and pLsLF-CTB-Fx3Pris were bombarded into tobacco and lettuce leaves, respectively. After 4-6 weeks, green shoots appeared and were subjected to three rounds of selection. Homoplasmy was confirmed by Southern blots. Transformed and untransformed tobacco DNA was digested with Afllll and probed with a 0.81-kb sequence complimentary to the trnI/trnA region of the native chloroplast genome. This produced a 4.2-kb fragment in the untransformed tobacco (FIG. 2a) and a 6.4-kb fragment in the transplastomic tobacco (FIG. 2b). Both transplastomic lines A and B showed only one hybridizing fragment of 6.4-kb size and did not show a fragment of the same size as the untransformed plant, indicating that all the chloroplast genomes had integrated the foreign gene and confirmed homoplasmy (FIG. 2c). Similarly, transformed and untransformed lettuce DNA was digested with Bglll which produced a 3.75-kb fragment in the untransformed lettuce (FIG. 3a) and a 6.3-kb fragment in the transplastomic lettuce (FIG. 3b). Only the 6.3-kb band was present in all transplastomic lines tested, confirming homoplasmy (FIG. 3c).

### 2.2 Detection and Quantification of Cleavable Proinsulin

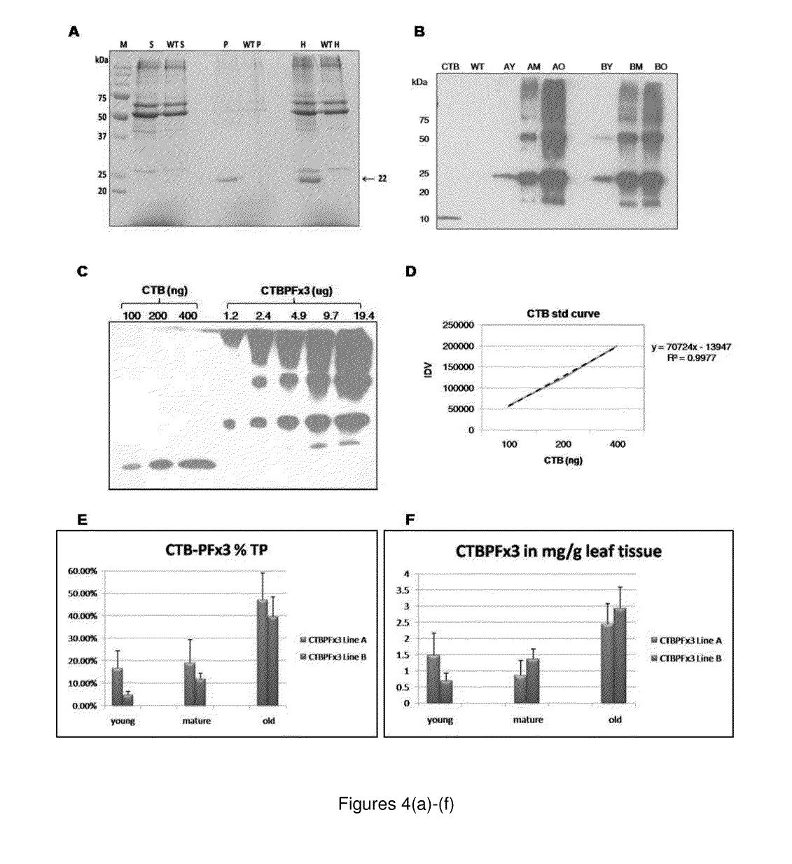

Coomassie staining and immunoblotting were used to determine whether the transgenic plants were expressing CTB-PFx3. Transplastomic and untransformed young, mature and old leaves were ground to a fine powder in liquid nitrogen, and protein was extracted with the addition of 300-500 μL plant extraction buffer. Young leaves are the new, light green, smaller leaves growing at the top of the plant. Mature leaves are large, dark green and grow towards the middle of the plant. Old leaves are the bottom-most senescent leaves that are beginning to turn brown. Samples were spun down, and the resulting supernatant, pellet and homogenate fractions were collected and run on a 12% SDS-PAGE gel (FIG. 4a). A polypeptide was seen prominently at 22 kDa in the pellet and homogenate fractions, but not in the supernatant, indicating that the CTB-PFx3 accumulated in the form of insoluble inclusion bodies. Immunoblots were performed using anti-CTB antibody to further confirm transgene expression. Young, mature and old homogenate samples were loaded in equal protein concentrations along with wild-type homogenate and recombinant CTB protein (FIG. 4b). A prominent band at 22 kDa represents the monomer size of the CTB-PFx3, but it is clear that the protein forms dimers, trimers, tetramers, pentamers and multimers of 44 kDa, 66 kDa, 88 kDa and 110 kDa sizes. Varying dilutions of the CTB-PFx3 homogenate were loaded onto a 12% SDS-PAGE gel along with known concentrations of recombinant CTB protein, and spot densitometry was performed in order to quantify expression levels (FIG. 4c). Different concentrations of CTB were loaded onto the gel in order to generate a standard curve (FIG. 4d). CTB-PFx3 was expressed in levels up to 47% in old leaves (FIG. 4e) and as much as 2.92 mg/g leaf tissue (FIG. 4f).

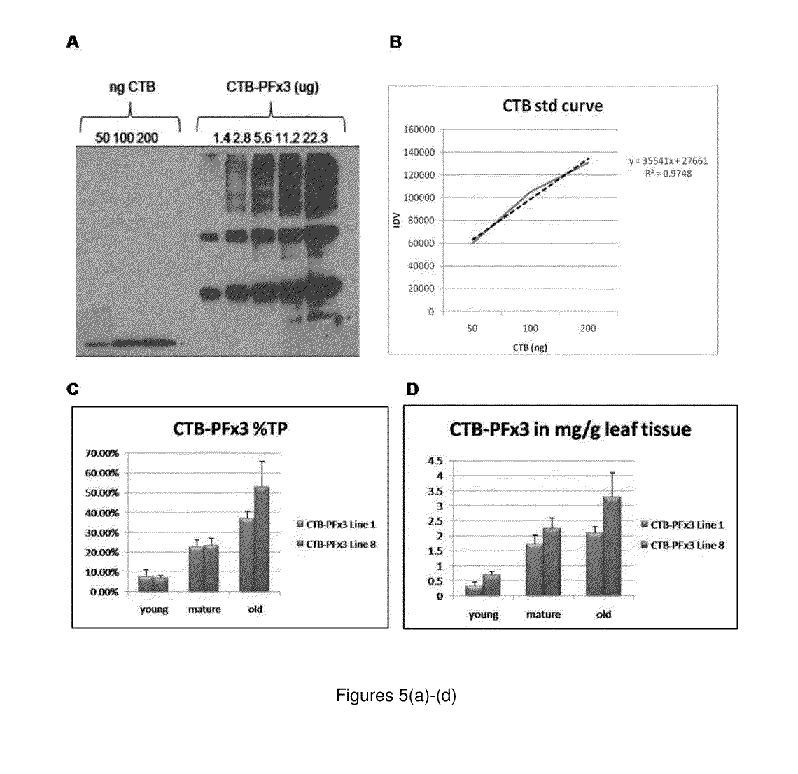

Transgenic lettuce plants were prepared in the same way as tobacco, and varying dilutions of the samples were loaded onto a 12% SDS-PAGE gel for spot densitometric analysis of protein concentration (FIG. 5a). Different concentrations of recombinant CTB protein were loaded onto the gel in order to create a standard curve (FIG. 5b). Of the four lines tested, CTB-PFx3 was found to account for up to 53% of the total leaf protein (FIG. 5c) and as much as 3.28 mg/g leaf tissue (FIG. 5d). The majority of expressed protein was found in the old leaves, and expression levels were comparable to levels observed in tobacco.

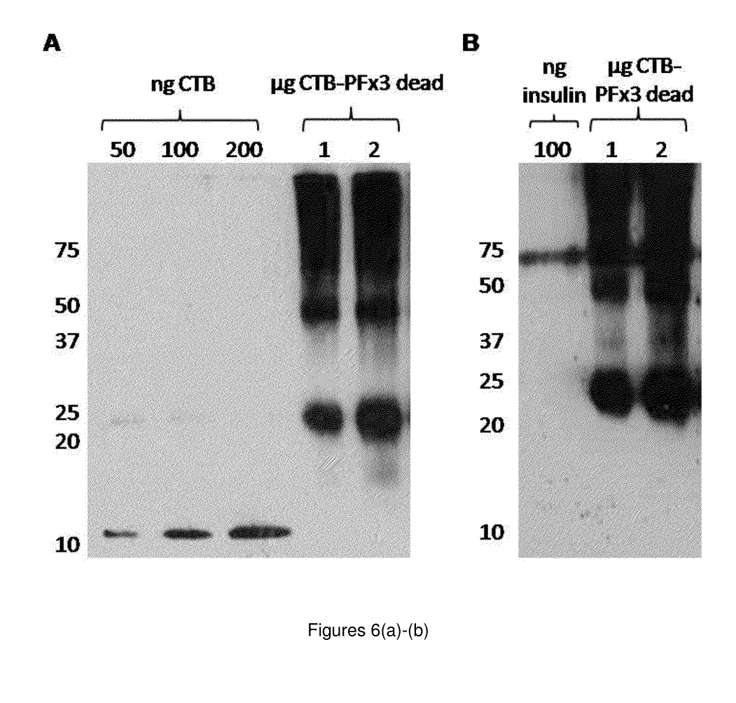

Additionally, owing to the high levels of expression observed in old leaves, senescent and dried lettuce leaves were tested to investigate stability of CTB-PFx3. Senescent leaves that were completely brown and dry were harvested from transplastomic lettuce plants, and protein was extracted as previously described. Leaf homogenate ranging from 0.125 to 2 μg total leaf protein was loaded on a 12% SDS-PAGE gel and probed with CTB or insulin antibodies (FIG. 6, a and b). High levels of CTB-PFx3 expression, up to 39% total leaf protein, were detected even in senescent lettuce leaf material, further demonstrating that owing to the insolubility of CTB-PFx3, it is highly stable and protected from proteolytic degradation.

### 2.3 Solubilization and Furin Cleavage of Proinsulin

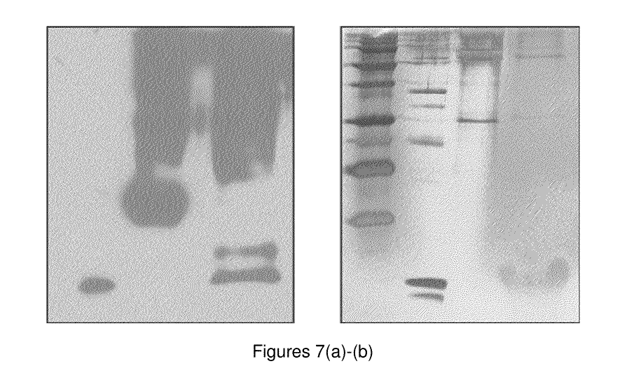

To prepare the CTB-PFx3 for purification and cleavage assays, it was feasible to remove this protein as inclusion bodies and convert into a soluble form. Previously, in our laboratory, insoluble inclusion bodies of human serum albumin (HSA) expressed in tobacco chloroplasts were solubilized and successfully purified (Fernandez-San Millan et al., 2003). Solubilization was achieved using high quantities of guanidine hydrochloride (Gu-HCl) and DTT. DTT was added to pellet resuspended in plant extraction buffer containing 6 m Gu-HCl. No CTB-PFx3 was found in the supernatant fraction after extraction, and this protein was entirely seen in the pellet fraction. Addition of 100 mm DTT achieved some solubility, but addition of 200 or 300 mm DTT achieved almost complete solubility of CTB-PFx3. Therefore, 300 mm DTT was used for all subsequent solubilizations. After solubilization, the denatured protein was refolded in 20 mm sodium phosphate pH 7.8 and 500 mm NaCl. To determine whether the engineered cleavage sites were functional, a furin cleavage assay was performed (FIG. 7a). Upon addition of furin and immunoblot visualization with CTB antibody, it is seen that the 22 kDa polypeptide representing the size of the CTB-PFx3 monomer disappears, and a polypeptide is seen at ˜11 kDa representing CTB alone as well as a polypeptide at ˜14 kDa which most likely represents an incomplete cleavage between CTB and proinsulin consisting of CTB fused to the B-chain of proinsulin, with cleavage occurring at the B-chain/C peptide junction. The proinsulin 2-3 kDa cleavage products were not detected by immunoblot with insulin antibody most likely because of the small size of the protein. Silver stain was then performed to visualize the 2-3 kDa products (FIG. 7b). In this gel, the monomer of CTB-PFx3 is seen to decrease after addition of furin, and a large band appears at the 2-3 kDa size. One large band is visualized because the A-chain, C peptide and B-chain cleavage products of proinsulin are 2 kDa, 2.5 kDa and 3 kDa, respectively, which are too close in size to be resolved independently.

### 2.4 Purification and Enrichment of Proinsulin Using Nickel Affinity Chromatography

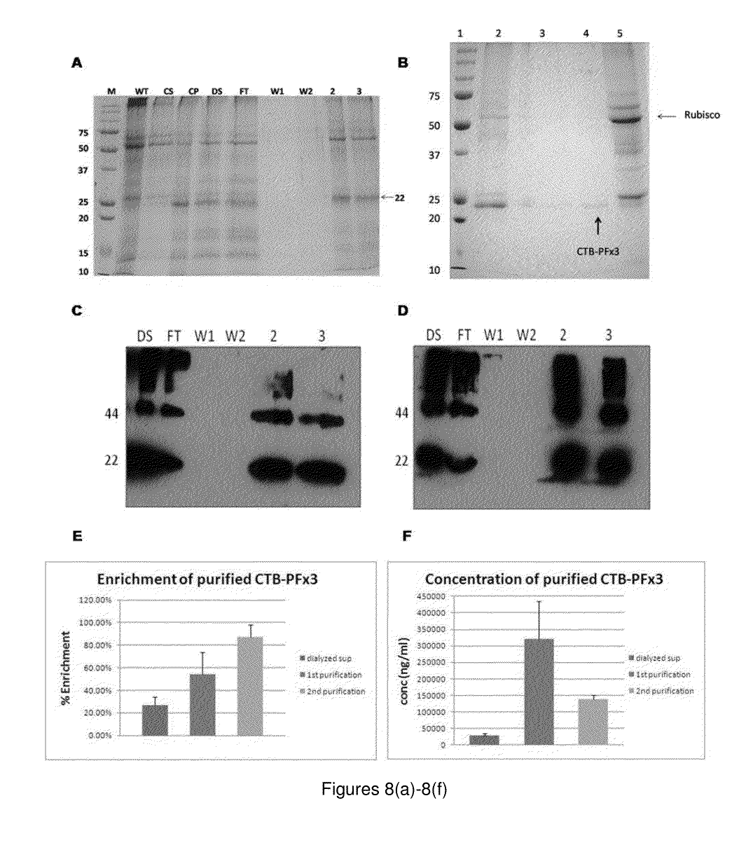

Solubilized and dialysed plant extracts containing CTB-PFx3 were passed over a Ni-NTA column to facilitate purification. Fractions were eluted and run on an SDS-PAGE gel and visualized by coomassie staining (FIG. 8a). The 55-kDa Rubisco large subunit is seen prominently in the wild-type sample. This is typical as Rubisco is the most abundant protein in plants and probably the most abundant protein on earth (Jensen, 2000). The 22-kDa band of CTB-PFx3 is seen prominently in the control pellet (CP), but not in the control supernatant (CS), again showing that the protein is in the form of inclusion bodies. After solubilization and dialysis, CTB-PFx3 is detected in the dialysed supernatant fraction (DS). Approximately 30 mL of dialysed supernatant was passed over the Ni-NTA column. No protein is seen to elute in the wash fractions (W1 and W2). The two eluted fractions with the highest optical density (OD) at 280 nm were run on the gel. The 22 kDa polypeptide representing CTB-PFx3 is seen prominently along with Rubisco, but it is seen that the contaminating protein bands are much less abundant; therefore, total CTB-PFx3 protein content was purified and compared with the starting material.

Purification was repeated in order to achieve a greater level of purity by removing Rubisco. In order to enhance purification, old leaves were prepared in the same way as in the previous purification as they contain less Rubisco, and samples were run over a Ni-NTA column. After purification, additional Rubisco was removed using sodium phytate precipitation (Krishnan and Natarajan, 2009), and samples were loaded on an SDS-PAGE gel for coomassie staining (FIG. 8b).

The resultant purified fractions were loaded in equal quantity onto an SDS-PAGE gel for immunoblot analysis along with the dialysed supernatant, flow through and wash fractions. Blots were probed with either anti-CTB antibody (FIG. 8c) or anti-insulin antibody (FIG. 8d). In both blots, the purified CTB-PFx3 is shown to be highly enriched compared to the dialysed supernatant. Additionally, the CTB-PFx3 monomer is enhanced, which would provide the functional, cleavable proinsulin. Densitometric analysis showed that the original dialysed supernatant contained 27% CTB-PFx3, whereas after the first purification, this increased to an average of 57% purity (range determined by densitometry to be between 42% and 72%), a nearly twofold increase. After the second step of purification followed by sodium phytate precipitation, the sample was shown to be about 87% pure (range determined by densitometry to be between 75% and 98%), over a threefold increase from the original dialysed supernatant and a 1.5-fold increase from the first purification (FIG. 8e). The starting concentration of the CTB-PFx3 in the dialysed supernatant was ˜30 000 ng/mL (0.03 mg/mL) which increased to ˜320 000 ng/mL (0.32 mg/mL) after the first purification and to ˜140 000 ng/mL (0.14 mg/mL) after the second purification representing a total enrichment greater than 10-fold and 4.5-fold, respectively (FIG. 8f). The lower concentration in the second purification is most likely due to the fact that less total sample volume was loaded onto the column resulting in a lower concentration but achieving higher purity.

### 2.5 Functional Evaluation of Cleavable Proinsulin

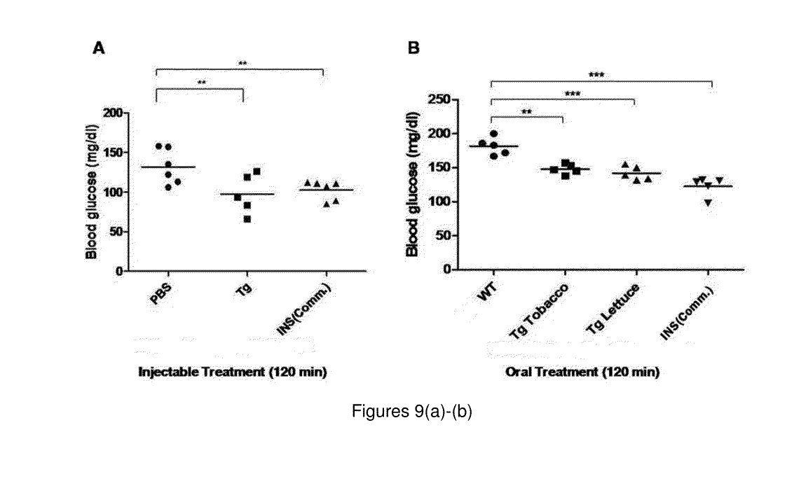

CTB-PFx3 purified from transplastomic tobacco as well as commercially produced purified insulin (Roche, Basel, Switzerland) was injected into female C57BL/6 mice along with PBS as a negative control. Blood glucose measurements were made by tail vein bleed before intraperitoneal (IP) injection and at 15-, 30-, 60-, 120- and 180-min time intervals. Another injection was given after the 180-min time point, and blood glucose measurements were again made at 15-, 30-, 60-, 120- and 180-min time points after the second injection. Blood glucose levels were found to be significantly lower than PBS control in all groups at the 120-min time point after the second injection (FIG. 9a), similar to commercial insulin.

Additionally, transplastomic lettuce and tobacco plants along with untransformed lettuce were delivered to mice by oral gavage. Commercially produced purified insulin was injected as a control. Blood glucose measurements were taken by tail vein bleed before gavage or injection and at 15-, 30-, 60-, 120- and 180-min time intervals. Another dose was given after the 180-min time point, and blood glucose measurements were again taken at 15-, 30-, 60- and 120-min time points after the second dose. Blood glucose levels were found to be significantly lower than untransformed control in all groups at the 120-min time point after the second injection (FIG. 9b). Food pellets were removed from cages for the duration of both studies.

### 3. DISCUSSION

Existing treatments for type 1 diabetes are painful, costly, and currently available insulin does not include the C-peptide. Here, CTB-proinsulin was expressed with three furin cleavage sites in both tobacco and lettuce chloroplasts in order to produce insulin that may be processed in most cells in the body. This will significantly reduce the cost of production, as well as facilitate the possibility of delivering the C-peptide, which should aid in the treatment of diabetic complications. One acre of farm land can yield about 40 metric tons of tobacco with three harvests (Arlen et al., 2007) or 11 metric tons of lettuce biomass in a single harvest. Assuming the expression level of CTB-PFx3 is around 3 mg/g of leaf tissue, and taking into consideration a 50% loss of protein during the purification process, this could yield up to 20 million daily doses of insulin. However, the efficiency of refolding would actually determine yield of functional protein, and this has not been quantified in this study. It is estimated that 220 million people worldwide have diabetes, 10% of these having type 1 diabetes. Therefore, one acre of biomass could produce enough insulin to treat a large type 1 diabetic population, which would be a major economic advantage.

There are many disadvantages to insulin injections which are currently the most widespread form of administration. These include, pain, itching, allergy and lipodystrophy at the injection site. Additionally, these disadvantages often cause noncompliance of patients with their insulin regimen. Therefore, alternative approaches to insulin delivery are necessary to help diabetic populations meet their medical needs. Some of the recent approaches to address these problems include buccal spray insulin, inhalable insulin, oral anti-diabetic drugs and oral delivery of insulin. Buccal spray insulin is absorbed by the mucosa of the mouth and has a faster onset than injectable insulin. Limitations of this approach include a shorter duration of action and a dosage requirement that is 5-7 times higher than injectable insulin as well as mild side effects such as dizziness (Pozzilli et al., 2010). Inhalable insulin faces similar challenges. The FDA approved inhalable insulin, Exubera, was pulled off the market because of poor sales and adverse side effects such as reduced lung function (Siekmeier and Scheuch, 2008). Oral anti-diabetic drugs are used mainly to treat type 2 diabetics as they improve insulin production, so there is still a need to deliver functional insulin to people with type 1 diabetes and type 2 diabetics who require insulin. The main challenges that arise in advancing the concept of oral delivery of insulin are protection from enzymatic degradation in the stomach and efficient absorption into the gut-associated lymphoid tissue (GALT, Agarwal and Khan, 2001). Approaches to overcome these challenges include encapsulation of insulin using nanoparticles and the delivery of insulin along with a protease inhibitor (Iyer et al., 2010). A novel approach to circumvent these obstacles is the expression of insulin in plant cells.

Insulin has never been produced without a fusion protein, as it is highly unstable and is prone to N-terminal degradation (Boothe et al., 2010). Therefore, proinsulin was fused with CTB in order to protect the N-terminus of insulin from degradation as well as provide a means to facilitate purification and uptake of orally delivered insulin into the GALT. CTB binds to GM1 receptors in the lumen of the gut (Tsuji et al., 1995; de Haan et al., 1998), and it has been shown that fusion to the C-terminus of CTB is ideal for pentamerization and GM1 binding (Liljeqvist et al., 1997; Limaye et al., 2006). Recently, CTB fused with blood clotting factor IX was successfully expressed and delivered orally to aid in treatment of haemophilia B. After delivery, the antigen was found in different tissues of the GALT, specifically the ileum, Peyer's patches, liver and plasma within two hours (Verma et al., 2010). In order to successfully obtain purified, functional insulin, the CTB-PFx3 must be solubilized in high quantities of reducing agent. This was achieved by using 6 m GuHCl and 300 mm DTT. After solubilizing, it was necessary to refold the denatured protein. This was achieved by dialysis in 20 mm sodium phosphate buffer containing 500 mm NaCl. The refolded CTB-PFx3 was then run over the Ni²⁺ column. Proper refolding was necessary in order to achieve purification because the lack of a His-tag made it necessary to use the refolded CTB moiety to bind to the Ni²⁺ column. CTB contains three histidine residues that are in close enough proximity when properly folded, which is enough to allow binding to anion exchange resins such as Ni²⁺ or Co²⁺ (Dertzbaugh and Cox, 1998). This way, purification of the CTB-PFx3 is facilitated by the CTB fusion. Additionally, purification is enhanced by the fact that a majority of the plant proteins are discarded with the supernatant, thus the initial sample to be purified contains only those proteins that were insoluble, eliminating a majority of endogenous plant soluble proteins. When expressed in plants, the insulin is also protected from enzymatic degradation in the stomach via bioencapsulation (Limaye et al., 2006; Davoodi-Semiromi et al., 2010; Verma et al., 2010). Therefore, advancing the concept of oral delivery of insulin by expressing it in plant cells should reduce its cost, facilitate purification and processing, eliminate the need for cold storage and simplify transportation and delivery (Daniell et al., 2009a).

Additionally, none of the alternative or currently used approaches to insulin delivery include the C-peptide. C-peptide is generally used as a marker to determine the extent of insulin release in patients. A large portion of diabetic patients who undergo extensive insulin therapy to maintain normal blood glucose levels still develop long-term complications such as neuropathy and nephropathy. C-peptide has been shown to alleviate these complications in various studies, and this suggests that diabetic patients should receive supplemental C-peptide in addition to insulin (Hills and Brunskill, 2009). For these reasons, cleavable proinsulin has been created, which is able to be processed into properly folded and functional insulin as shown by functionality studies in mice. Furin cleavage sites were chosen owing to the fact that furin is an ubiquitous protease found in all human cells and tissue types. There has been work with furin protease in previous animal studies involving oral tolerance, and no adverse immunogenic effects have been seen (Verma et al., 2010). Furthermore, furin cleavage has been used in several studies to process human proinsulin to achieve safe and long-term expression (Suda et al., 2006). Upon cleavage, C-peptide is available for circulation, where it may interact with its G-protein-coupled receptor and in turn activate the Na+/K+ ATPase and MAPK pathways, which are partly responsible for the beneficial effects seen in alleviating diabetic complications. This is the first time that the complete proinsulin gene has been expressed in plants with the possibility of in vivo processing outside the pancreas, into functional insulin and C-peptide.

Upon hyper-expression of cleavable proinsulin in tobacco and lettuce chloroplasts, expression in the form of insoluble inclusion bodies similar to insulin inclusion bodies observed in E. coli (Williams et al., 1982) or plants (Boothe et al., 2010) was noticed. The functionality of CTB stems from its ability to pentamerize in order to bind to GM1 receptors (Liljeqvist et al., 1997) and has been demonstrated to form functional pentamers in chloroplasts (Daniell et al., 2001; Limaye et al., 2006; Verma et al., 2010). Additionally, insulin and proinsulin may form self-aggregates of monomers, dimers, tetramers and hexamers (Pekar and Frank, 1972), which may further contribute to their aggregation in chloroplasts. However, formation of pentamers, multimeric forms and inclusion bodies caused the proinsulin to be highly stable by conferring protection from proteases. The majority of proinsulin accumulation was seen in older leaves as opposed to mature leaves, which is often observed among foreign (soluble) proteins expressed in chloroplasts (Tsuji et al., 1995; Koya et al., 2005; Arlen et al., 2008; Daniell et al., 2009b). This may account for the high levels of expression seen in old leaves, as proinsulin may be protected from proteases (Martin and Thimann, 1972; Vierstra, 1993). Stability of CTB-PFx3 was even seen in senescent and dried leaves that should facilitate harvest, field drying, storage and transportation of this valuable protein. This is the first report of stable accumulation of a therapeutic protein in dried senescent leaves.

Attempts made to determine protein quantification by means of enzyme-linked immunosorbent assay (ELISA) were unsuccessful because of the high quantity of protein aggregates, which either inhibited binding to the ELISA plate or reduced the ability of the CTB primary antibody from binding to the proper epitopes on the CTB molecule. Addition of DTT to the sample extraction buffer or boiling of samples prior to coating of the ELISA plate with CTB-PFx3 improved the detection of signal, but not enough to yield an accurate quantification. The denaturation of the CTB-PFx3 monomer and multimers during the immunoblot procedure most likely facilitated better binding of the CTB primary antibody, allowing access to the desired epitopes. For densitometry studies, only the monomer and dimer bands were used for the quantification, as the higher multimers tended to form less defined bands. Therefore, the percentages reported are likely an underestimation of the total expressed CTB-proinsulin.

Mice that were given oral CTB-PFx3 experienced changes in blood glucose levels, but levels were significantly lower at 120 min after the second dose when compared to mice given untransformed lettuce. This demonstrates that proinsulin is delivered to the circulation and is properly cleaved and folded. In a recently published study, blood clotting factor IX fused with CTB in the circulatory system 2 h after oral delivery (the earliest time point in this study) of CTB-FIX expressed in chloroplasts (Verma et al., 2010) was observed. Therefore, detection of plant-derived insulin activity in less than two hours is quite reasonable. At this point, the percentage of orally delivered protein absorbed by the GALT and delivered into the circulatory system has not been fully investigated, although several bioencapsulated chloroplast-derived therapeutic proteins have been orally delivered and shown to be fully functional. Additionally, injected CTB-PFx3 must be processed into CTB and insulin when compared with the commercial insulin, which is already in its correct functional conformation. However, the efficiency of refolding has not been quantified in our study. Therefore, determining the actual amount of CTB-PFx3 mice received and time required for processing would require further investigations. Future studies would investigate long-term effects of the C-peptide delivery on diabetic complications in addition to the effects of CTB-PFx3 on blood glucose levels. Although solubilization of multimers of proinsulin into monomers for effective delivery of functional insulin was investigated, a recent study has shown that injection of aggregates of insulin oligomers facilitates sustained and prolonged release, up to 140 days (Gupta et al., 2010).

### 4. EXPERIMENTAL PROCEDURES

### 4.1 Vector Construction

The CTB-PFx3 construct was created from pLD-5′UTR-CTB-Pins as previously described (Ruhlman et al., 2007). The proinsulin gene was modified in order to include furin cleavage sites in between the B-chain/C-peptide and the C-peptide/A-chain junctions as well as between the CTB/B-chain fusion site. The pLD-5′UTR-CTB-Pins vector was digested with NcoI and SmaI, and the resulting portion containing the 5′UTR was ligated into the pGEM-Pris(fx3) vector containing the mutated proinsulin. This was subsequently digested with EcoRI and NotI and subcloned into the pLD-g10-RecA vector, replacing the g10-RecA gene with the 5′Utr CTB-Pris(fx3) gene. The final vector was designated pLDutr-CTB-Fx3Pris. To create the lettuce vector, pLDutr-CTB-Fx3Pris was digested with SmaI and XbaI, and the resulting Fx3Pris fragment was ligated into the 12.13.9.11 CTB-GPGP-FIX vector containing the lettuce-specific psbA 3′ and 5′ UTRs, replacing the FIX gene. To create the final vector, the pLspsbACTB-Fx3 Pris was digested with NdeI and NotI and was cloned into pLsLFHPAG vector containing the lettuce flanking regions, and this vector was designated as pLsLF-CTB-Fx3Pris. After vector construction, the CTB-PFx3 genes were sequenced (GENEWIZ). Sequences were confirmed to be correct using BLAST search.

### 4.2 Bombardment and Selection

Bombardment was done using the Bio-Rad PDS-1000/He gene gun as described previously (Verma et al., 2008). Sterile tobacco leaves were placed abaxial side up, whereas sterile lettuce leaves were placed abaxial side down on Murashige and Skoog (MS) medium and were bombarded under sterile conditions using pLDutr-CTB-Fx3Pris or pLsLF-CTB-Fx3Pris coated with gold particles prepared according to (Kumar and Daniell, 2004). Bombarded leaf pieces were incubated in the dark for 2 days at room temperature, and then leaf segments were transferred to regeneration media of plants (RMOP) containing 500 mg/L spectinomycin for tobacco or regeneration media of lettuce (RMOL) containing 100 mg/L spectinomycin (Ruhlman et al., 2010). The appearance of green shoots was seen after 4-6 weeks. These shoots were allowed to grow, and the leaves were cut and subsequently transferred to RMOP or RMOL containing 500 mg/L or 100 mg/L spectinomycin, respectively, for the second round of selection. After new shoots formed, these were transferred for the third round of selection to MS containing 500 mg/L spectinomycin for tobacco or 100 mg/L spectinomycin for lettuce and allowed to establish roots. Plants were then transferred to the greenhouse.

### 4.3 Southern Analysis to Confirm Homoplasmy

Transgenic and untransformed tobacco DNA was digested using Afllll, and lettuce DNA was digested using Bglll in a reaction mixture containing 2 μL 10× buffer (New England Biolabs, Ipswich, Mass.), 3 μg plant genomic DNA, 2 μL BSA and 1 μL enzyme made up to 20 μL with dH2O. The reactions were incubated overnight at 37° C. Southern analysis was performed according to laboratory protocol (Kumar and Daniell, 2004). The entire 20 μL restriction digestion samples were separated on a 0.8% agarose gel at 50V for 4 h in Tris-acetic acid-EDTA (TAE) buffer. The gel was depurinated for 15 min in 0.25N HCl, rinsed two times for 5 min each in dH2O and soaked in transfer buffer (0.4N NaOH, 1 m HCl) for 20 min. All steps were performed with gentle rocking. Capillary transfer was allowed to take place over night. The next day, the membrane was rinsed with 2×SSC (0.3 m NaCl, 0.03 m Sodium Citrate) two times for 5 min each, and DNA was cross-linked to the membrane at 150 mJ in GS Gene Linker (Bio-Rad, Hercules, Calif.). Flanking sequence probe was generated by digesting the pUC-CT vector (Verma et al., 2008) with Bglll and BamHI in order to generate a 0.81-kb fragment corresponding to the trnI/trnA region of the native chloroplast genome. Probe was labelled with 32P using Ready-to-Go DNA labelling beads (General Electric, Fairfield, Mass.). Hybridization was carried out using Stratagene Quick-Hyb solution and protocol. Afterwards, the membrane was placed in a cassette and exposed to X-ray film at −80° C.

### 4.4 Protein Extraction and Quantitation

Young, mature and old leaf tissue was ground to a fine powder in liquid nitrogen using a mortar and pestle. Five hundred microlitres of plant extraction buffer (100 mm NaCl, 200 mm Tris-HCl pH 8.0, 0.1% Triton-X, 400 mm sucrose, and Roche complete mini protease inhibitor cocktail tablet) was added to 100 mg of frozen leaf tissue and vortexed for 10 min at 4° C. Plant tissue homogenates were further separated into supernatant and pellet fractions via centrifugation (20 000 g for 5 min at 4° C.), and the pellet was resuspended in 500 μL plant extraction buffer for analysis. Total leaf protein concentration was determined using the Bio-Rad protein assay dye reagent concentrate. Dye was diluted 1:5 in water and filtered through Whatman filter paper. The standard curve was made using 0.4 mg/mL BSA serially diluted down to 0.006 mg/mL. Plant homogenate, supernatant and pellet samples were diluted in water 1:5, 1:10, 1:20 and 10 μL of sample, and BSA standard was loaded in duplicate to a 96-well plate. Diluted Bio-Rad dye (200 μL) was added to each sample and standard and the plate was read on a plate reader at 595 nm.

### 4.5 SDS-PAGE and Immunoblot Analysis

Homogenate, supernatant and pellet samples were diluted in 2× sample loading buffer (3.55 mL dH2O, 1.25 mL 0.5 m Tris-HCl pH 6.8, 2.5 mL glycerol, 2 mL 10% SDS, 0.2 mL 0.5% bromophenol blue) and loaded onto a 12% SDS-PAGE (Bio-Rad) gel. Samples were separated at 100-150V until the dye front reached the bottom of the gel. After separation, the gel was either stained with Coomassie Brilliant Blue R-250 (Bio-Rad) or transferred to a nitrocellulose membrane (Bio-Rad) for immunoblot analysis. Transfer was carried out using a Bio-Rad transfer cassette at 85V for 1 h. Membranes were briefly rinsed in dH2O and blocked in PBS-Tween-20+3% dry milk (PTM) for 30 min to 1 h. CTB-PFx3 was detected using anti-CTB primary antibody (Sigma, St. Louis, Mo.) diluted 1:4000 for 1.5 h followed by goat anti-rabbit IgG-HRP (Southern Biotech, Birmingham, Ala.) at a dilution of 1:5000 for 1 h. Proteins were subsequently detected on autoradiography film using SuperSignal West Pico Chemiluminescent Substrate (Thermo Scientific, Waltham, Mass.).

### 4.6 Densitometric Quantification of CTB-Proinsulin

Immunoblots for quantification were performed using known concentrations of CTB protein standard (Sigma) of 25, 50, 100, 200 and 400 ng in order to create a standard curve. Total protein concentration was determined via the Bradford assay (Bio-Rad). Varying concentrations of CTB-PFx3 plant homogenate were directly compared to the standard curve using Alphaimager and Alphaease FC software. The percentage of CTB-PFx3 relative to the concentration of total leaf protein and mg CTB-PFx3 per gram leaf tissue were calculated according to the formula published in (Verma et al., 2008).

### 4.7 Protein Solubilization and Furin Cleavage

CTB-PFx3 homogenate was separated into supernatant and pellet fractions. The pellet fraction containing the CTB-PFx3 in an insoluble form was resuspended in 1.5 mL of plant extraction buffer per 100 mg ground plant tissue along with 100-300 mm dithiothreitol (DTT, Promega Fitchburg, Wis.) and 6 m guanidine hydrochloride (Gu-HCl, Sigma). Samples were rocked for 16-18 h at 4° C. The samples were then centrifuged at 14 000 rpm for 5 min at 4° C., and the supernatant containing soluble CTB-PFx3 was separated from the pellet fraction. The samples were subsequently dialysed using Spectra/Por molecularporous membrane tubing (Fisher Scientific, Waltham, Mass.) in 20 mm sodium phosphate pH 7.8, 500 mm NaCl overnight at 4° C. For cleavage with furin protease, 1 μg of dialysed sample was incubated with 100 mm HEPES pH 7.5, 0.5% Triton X-100, 1 mm CaCl2, 1 mm 2-mercaptoethanol, and 2 units furin (New England Biolabs) at 25° C. for 16 h.

### 4.8 Affinity Purification

After solubilization and dialysis of the CTB-PFx3, the resulting supernatant was passed over a column (Clontech, Mountain View, Calif.) consisting of 2 mL Ni-NTA Agarose (Qiagen, Venlo, the Netherlands) which was equilibrated with 20 mm sodium phosphate pH 7.8 and 500 mm NaCl (Binding buffer). The column was subsequently washed with 30 mL binding buffer and then with 30 mL wash buffer (20 mm sodium phosphate pH 6.0 and 500 mm NaCl). Samples were eluted from the column in 1 mL fractions using 10 mL elution buffer (20 mm sodium phosphate pH 6.0, 500 mm NaCl, and 250 mm imidazole). All steps were carried out at a flow rate of 0.5 mL/min. Fractions were assayed for protein concentration via absorbance units at 280 nm using the SmartSpec 3000 (Biorad) and Bradford assay to determine the concentration in mg/mL. Purified CTB-PFx3 (1 μg) was incubated with furin, and cut and uncut samples were run on a 16% Tricine-SDS gel. Samples were also subsequently run on 12% SDS-PAGE gel and subjected to coomassie stain and immunoblot in order to determine the percent purity.

### 4.9 Sodium Phytate Precipitation

Sodium phytate precipitation was performed to remove Rubisco from purified samples. Purified samples were treated with 10 mm CaCl2 and 10 mm sodium phytate (phytic acid sodium salt hydrate from Sigma) for 10 min at 37° C. Afterwards, samples were spun in a tabletop centrifuge at room temperature for 10 min at 14 000 rpm. Supernatant was removed, placed in a fresh tube and stored at −20° C. until use.

### 4.10 Silver Stain

Tricine-SDS gel was incubated in fixative (45% dH2O, 45% methanol, 10% acetic acid) for 40 min. The gel was then washed with 50% methanol 2× for 5 min each and then with dH2O 2× for 5 min each. Next, the gel was rocked in Hypo solution (20 mg Na2S2O3 in 100 mL dH2O) for exactly 1 min and then washed in dH2O 3× for 5 min each. Silver solution (200 mg AgNO3 and 100 μL formaldehyde in 100 mL dH2O) was added and the gel was rocked for 30 min. The gel was washed in dH2O 4× for 5 min each and then developer solution (6 g Na2CO3, 100 μL of hypo, 100 μL formaldehyde in 300 mL dH2O) was added. The gel was vigorously shaken in developer solution until protein bands appeared, and then the reaction was stopped by addition of 5% acetic acid.

### 4.11 Animal Studies to Determine Functionality

All procedures performed in this study are based on an approved protocol in accordance with UCF-IACUC. Female C57BL/6 mice weighing 17-20 g each were purchased from the Jackson Laboratories (Bar Harbor, Me.). Mice were divided into four groups with six mice per group. Samples were prepared for delivery via IP injection by dilution in sterile PBS. Commercially purified insulin and purified CTB-PFx3 derived from transgenic tobacco were normalized via immunoblot and subsequently diluted to give each mouse a dosage of 0.04 U/kg of commercially prepared insulin (Roche) and 8 μg/mouse of plant-derived purified CTB-PFx3. IP injections were delivered in a volume of 100 μL. Blood glucose levels were measured by bleeding of the tail vein before sample administration and again at 15, 30, 60, 120 and 180 min after sample administration (Accu-Chek, Aviva; Roche, Basel, Switzerland). A second injection was given after 180 min, and blood glucose measurements were taken at 15, 30, 60, 120 and 180 min after the second IP injection. Food pellets were removed for the duration of the study. For the oral study, female C57BL/6 mice were divided into four groups with five mice per group. Commercially purified insulin was diluted in sterile PBS and given at a dose of 0.04 U/kg. IP injections were delivered in a volume of 100 μL. Oral samples were prepared from material ground in liquid nitrogen and stored at −80° C. until the day of the experiment. Prior to gavage, 3 g of wild-type and transgenic leaf material were mixed with 500 μL 20×PBS and homogenized using a General Laboratory Homogenizer (GLH-2596, Omni International, Kennesaw, Ga.) for 5 min. Two hundred microlitres (˜200 mg) of leaf material containing approximately 0.5 mg CTB-PFx3 was administered orally to mice via a 20-gauge gavage needle. Blood serum was taken by bleeding of the tail vein before sample administration and again at 15, 30, 60, 120 and 180 min after sample administration. A second dose was given after 180 min, and blood glucose measurements were taken at 15, 30, 60 and 120 min after the second dose.

### REFERENCES

Agarwal, V. and Khan, M. (2001) Current status of the oral delivery of insulin. Pharm. Technol., October, 76-90.
American Diabetes Association (2008) Economic costs of diabetes in the U.S. in 2007. Diabetes Care, 31, 596-615.
Arakawa, T., Yu, J., Chong, D. K., Hough, J., Engen, P. C. and Langridge, W. H. (1998) A plant-based cholera toxin B subunit-insulin fusion protein protects against the development of autoimmune diabetes. Nat. Biotechnol., 16, 934-938.
Arlen, P. A., Falconer, R., Cherukumilli, S., Cole, A., Cole, A. M., Oishi, K. K. and Daniell, H. (2007) Field production and functional evaluation of chloroplast-derived interferon-alpha2b. Plant Biotechnol. J., 5, 511-525.
Arlen, P. A., Singleton, M., Adamovicz, J. J., Ding, Y., Davoodi-Semiromi, A. and Daniell, H. (2008) Effective plague vaccination via oral delivery of plant cells expressing F1-V antigens in chloroplasts. Infect. Immun., 76, 3640-3650.
Bally, J., Paget, E., Droux, M., Job, C., Job, D. and Dubald, M. (2008) Both the stroma and thylakoid lumen of tobacco chloroplasts are competent for the formation of disulphide bonds in recombinant proteins. Plant Biotechnol. J., 6, 46-61.
Boothe, J., Nykiforuk, C., Shen, Y., Zaplachinski, S., Szarka, S., Kuhlman, P., Murray, E., Morck, D. and Moloney, M. M. (2010) Seed-based expression systems for plant molecular farming. Plant Biotechnol. J., 8, 588-606.
Centers for Disease Control and Prevention (2008) National diabetes fact sheet: general information and national estimates on diabetes in the United States, 2007. Atlanta, Ga.: U.S. Department of Health and Human Services, Centers for Disease Control and Prevention.
Daniell, H. (2007) Transgene containment by maternal inheritance: effective or elusive? Proc. Natl. Acad. Sci. USA, 104, 6879-6880.
Daniell, H., Lee, S.-B., Panchal, T. and Wiebe, P. O. (2001) Expression of the Native Cholera Toxin B Subunit Gene and Assembly as Functional Oligomers in Transgenic Tobacco Chloroplasts. J. Mol. Biol., 311, 1001-1009.
Daniell, H., Singh, N., Mason, H. and Streatfield, S. J. (2009a) Plant-made vaccine antigens and biopharmaceuticals. Trends Plant Sci., 14, 669-679.
Daniell, H., Ruiz, G., Denes, B., Sandberg, L. and Langridge, W. (2009b) Optimization of codon composition and regulatory elements for expression of human insulin like growth factor-1 in transgenic chloroplasts and evaluation of structural identity and function. BMC Biotechnol., 9, 33.
Daniell H. (1997) Transformation and foreign expression in plants mediated by microprojectile bombardment. Methods in Molecular Biology. 62: 463-489.
Davoodi-Semiromi, A., Samson, N. and Daniell, H. et al. (2009) The green vaccine: a global strategy to combat infectious and autoimmune diseases. Hum. Vaccin., 5, 488-493.
Davoodi-Semiromi, A., Schreiber, M., Nalapalli, S., Verma, D., Singh, N. D., Banks, R. K., Chakrabarti, D. and Daniell, H. (2010) Chloroplast-derived vaccine antigens confer dual immunity against cholera and malaria by oral or injectable delivery. Plant Biotechnol. J., 8, 223-242.
Dertzbaugh, M. T. and Cox, L. (1998) The affinity of cholera toxin for Ni2+ ion. Protein Eng., 11, 577-581.
Ekberg, K., Brismar, T., Johansson, B. L., Jonsson, B., Lindström, P. and Wahren, J. (2003) Amelioration of sensory nerve dysfunction by C-Peptide in patients with type 1 diabetes. Diabetes, 52, 536-541.
Fernandez-San Millan, A., Minego-Castel, A., Mingo-Castel, A., Miller, M. and Daniell, H. (2003) A chloroplast genetic approach to hyper-express and purify human serum albumin, a protein highly susceptible to proteolytic degradation. Plant Biotechnol. J., 1, 71-79.
Fujimoto, K., Sasaki, T., Nemoto, M., Nakai, N., Sakai, K., Yamasaki, K., Hiki, Y., Ohashi, T., Eto, Y. and Tajima, N. (2005) Enhanced insulin secretion from engineered 3T3-L1 preadipocytes by induction of cellular differentiation. Mol. Cell. Biochem., 268, 1-8.
Giannini, C., Mohn, A. and Chiarelli, F. et al. (2009) Technology and the issue of cost/benefit in diabetes. Diabetes Metab. Res. Rev., 25(Suppl 1), S34-44.
Groskreutz, D. J., Sliwkowski, M. and Gorman, C. M. (1994) Genetically engineered proinsulin constitutively processed and secreted as mature, active insulin. J. Biol. Chem., 269, 6241-6245.
Gupta, S., Chattopadhyay, T., Singh, M. P. and Surolia, A. (2010) Supramolecular insulin assembly II for a sustained treatment of type 1 diabetes mellitus. Proc. Natl. Acad. Sci. USA, 107, 13246-13251.
de Haan, L., Verweij, W., Agsteribbe, E. and Wilschut, J. (1998) The role of ADP-ribosylation and GM1-binding activity in the mucosal immunogenicity and adjuvanticity of the Escherichia coli heat-labile enterotoxin and Vibrio cholerae cholera toxin. Immunol. Cell Biol., 76, 270-279.
Hills, C. E. and Brunskill, N. (2009) Cellular and physiological effects of C-peptide. Clin. Sci. (Lond), 116, 565-574.
Iyer, H., Khedkar, A. and Verma, M. (2010) Oral insulin—a review of current status. Diabetes Obes. Metab., 12, 179-185.
Jensen, R. (2000) Activation of Rubisco regulates photosynthesis at high temperature and CO2. Proc. Natl. Acad. Sci., 97, 12937-12938.
Johansson, B. L., Borg, K., Fernqvist-Forbes, E., Kernell, A., Odergren, T. and Wahren, J. (2000) Beneficial effects of C-peptide on incipient nephropathy and neuropathy in patients with Type 1 diabetes mellitus. Diabet. Med., 1, 181-189.
Kjeldsen, T. (2000) Yeast secretory expression of insulin precursors. Appl. Microbiol. Biotechnol., 54, 277-286.
Koya, V., Moayeri, M., Leppla, S. H. and Daniell, H. (2005) Plant-based vaccine: mice immunized with chloroplast-derived anthrax protective antigen survive anthrax lethal toxin challenge. Infect. Immun., 73, 8266-8274.
Krishnan, H. B. and Natarajan, S. (2009) A rapid method for depletion of Rubisco from soybean (Glycine max) leaf for proteomic analysis of lower abundance proteins. Phytochemistry, 70, 1958-1964.
Kumar, S, and Daniell, H. (2004) Engineering the chloroplast genome for hyperexpression of human therapeutic proteins and vaccine antigens. Methods Mol. Biol., 267, 365-383.
Lee, S. B., Li, B., Jin, S, and Daniell, H. (2011) Expression and characterization of antimicrobial peptides Retrocyclin-101 and Protegrin-1 in chloroplasts to control viral and bacterial infections. Plant Biotechnol. J. doi:10.1111/j.1467-7652.2010.00538.x
Levinson, P. D. (2003) Eighty years of insulin therapy: 1922-2002. Med. Health R. I., 86, 101-106.
Li, D., O'Leary, J., Huang, Y., Huner, N. P., Jevnikar, A. M. and Ma, S. (2006) Expression of cholera toxin B subunit and the B chain of human insulin as a fusion protein in transgenic tobacco plants. Plant Cell Rep., 25, 417-424.
Liljeqvist, S., Stahl, S., Andréoni, C., Binz, H., Uhlén, M. and Murby, M. (1997) Fusions to the cholera toxin B subunit: influence on pentamerization and GM1 binding. J. Immunol. Methods, 210, 125-135.
Limaye, A., Koya, V., Samsam, M. and Daniell, H. (2006) Receptor-mediated oral delivery of a bioencapsulated green fluorescent protein expressed in transgenic chloroplasts into the mouse circulatory system. FASEB J., 20, E37-E46.
Martin, C. and Thimann, K. (1972) The role of protein synthesis in the senescence of leaves: I. The formation of protease. Plant Physiol., 49, 64-71.
Oey, M., Lohse, M., Scharff, L. B., Kreikemeyer, B. and Bock, R. (2009) Plastid production of protein antibiotics against pneumonia via a new strategy for high-level expression of antimicrobial proteins. Proc. Natl. Acad. Sci. USA, 106, 6579-6584.
Pekar, A. H. and Frank, B. (1972) Conformation of proinsulin. A comparison of insulin and proinsulin self-association at neutral pH. Biochemistry, 11, 4013-4016.
Pozzilli, P., Raskin, P. and Parkin, C. G. (2010) Review of clinical trials: update on oral insulin spray formulation. Diabetes Obes. Metab., 12, 91-96.
Quesada-Vargas, T., Ruiz, O. and Daniell, H. (2005) Characterization of heterologous multigene operons in transgenic chloroplasts: transcription, processing, and translation. Plant Physiol., 138, 1746-1762.
Raab, R., Fezeu, L. and Mbanya, J. C. (2004) Cost and availability of insulin and other diabetes supplies: IDF survey 2003-2003. Diabetes Voice, 49, 24-29. Go here for SFX
Rebsomen, L., Khammar, A., Raccah, D. and Tsimaratos, M. (2008) C-peptide effects on renal physiology and diabetes. Exp. Diabetes Res., 2008, 1-5.
Ruhlman, T., Ahangari, R., Devine, A., Samsam, M. and Daniell, H. (2007) Expression of cholera toxin B-proinsulin fusion protein in lettuce and tobacco chloroplasts—oral administration protects against development of insulitis in non-obese diabetic mice. Plant Biotechnol. J., 5, 495-510.
Ruhlman, T., Verma, D., Samson, N. and Daniell, H. (2010) The role of heterologous chloroplast sequence elements in transgene integration and expression. Plant Physiol., 152, 2088-2104.
Shaw, J. A. M., Delday, M., Hart, A. W., Docherty, H. M., Maltin, C. A. and Docherty, K. (2002) Secretion of bioactive human insulin following plasmid-mediated gene transfer to non-neuroendocrine cell lines, primary cultures and rat skeletal muscle in vivo. J. Endocrinol., 172, 653-672.
Siekmeier, R. and Scheuch, G. (2008) Inhaled insulin—does it become reality? J. Physiol. Pharmacol., 59(Suppl 6), 81-113.
Sima, A. A. F. and Li, Z.-G. (2005) The effect of C-peptide on cognitive dysfunction and hippocampal apoptosis in type 1 diabetic rats. Diabetes, 54, 1497-1505.
Steiner, D. F. (1998) The proprotein convertases. Curr. Opin. Chem. Biol., 2, 31-39.
Suda, T., Katoh, M., Hiratsuka, M., Takiguchi, M., Kazuki, Y., Inoue, T. and Oshimura, M. (2006) Heat regulated production and secretion of insulin from a human artificial chromosome vector. Biochem. Biophys. Res. Commun., 340, 1053-1061.
Swartz, J. R. (2001) Advances in Escherichia coli production of therapeutic proteins. Curr. Opin. Biotechnol., 12, 195-201.
Tatake, R. J., O'neill, M. M., Kennedy, C. A., Reale, V. D., Runyan, J. D., Monaco, K. A., Yu, K., Osborne, W. R., Barton, R. W. and Schneiderman, R. D. (2007) Glucose-regulated insulin production from genetically engineered human non-beta cells. Life Sci., 81, 1346-1354.
Taylor, N. A., Van De Ven, W. and Creemers, J. W. (2003) Curbing activation: proprotein convertases in homeostasis and pathology. FASEB J., 17, 1215-1227.
Thomas, G. (2002) Furin at the cutting edge: from protein traffic to embryogenesis and disease. Nat. Rev., 3, 753-766.
Tsuji, T., Watanabe, K. and Miyama, A. (1995) Monomer of the B Subunit of Heat-Labile Enterotoxin from Enterotoxigenic Escherichia coli Has Little Ability to Bind to GM1 Ganglioside Compared to Its Coligenoid. Microbiol. Immunol., 39, 817-819.
Verma, D. and Daniell, H. (2007) Chloroplast Vector Systems for Biotechnology Applications. Plant Physiol., 145, 1129-1143.
Verma, D., Samson, N., Koya, V. and Daniell, H. (2008) A protocol for expression of foreign genes in chloroplasts. Nat. Protoc., 3, 739-758.
Verma, D., Moghimi, B., LoDuca, P. A., Singh, H. D., Hoffman, B. E., Herzog, R. W. and Daniell, H. (2010) Oral delivery of bioencapsulated coagulation factor IX prevents inhibitor formation and fatal anaphylaxis in hemophilia B mice. Proc. Natl. Acad. Sci. USA, 107, 7101-7106.
Vierstra, R. D. (1993) Protein Degradation in Plants. Annu. Rev. Plant Physiol. Plant Mol. Biol., 44, 385-410.
Wahren, J. (2004) “C-peptide: new findings and therapeutic implications in diabetes. Clin. Physiol. Funct. Imaging, 24, 180-189.
Wahren, J., Ekberg, K. and Jörnvall, H. (2007) C-peptide is a bioactive peptide. Diabetologia, 50, 503-509.
Williams, D. C., Van Frank, R. M., Muth, W. L. and Burnett, J. P. (1982) Cytoplasmic inclusion bodies in Escherichia coli producing biosynthetic human insulin proteins. Science, 215, 687-689.

The references cited herein, patent or non-patent related, are each incorporated by reference herein. While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

What is claimed is:

1. A cleavable proinsulin construct comprising:
a cholera toxin B (CTB) subunit fused to a proinsulin subunit; and
at least one furin cleavage site on the cleavable proinsulin construct.

2. The cleavable proinsulin construct of claim 1, wherein the proinsulin subunit comprises a C peptide

3. The cleavable proinsulin construct of claim 2, wherein the proinsulin subunit further comprises an A chain and a B chain, and wherein the C peptide is disposed between the A chain and the B chain.

4. The cleavable proinsulin construct of claim 2, wherein the at least one furin cleavage site comprises a pair of furin cleavage sites flanking each side of the C-peptide.

5. The cleavable proinsulin construct of claim 1, wherein the at least one furin cleavage site comprises a furin cleavage site located at a junction between the CTB subunit and the proinsulin subunit.

6. The cleavable proinsulin construct of claim 5, further comprising a GPGP (SEQ ID NO: 2) hinge sequence between the CTB sequence and the furin cleavage site.

7. The cleavable proinsulin construct of claim 1, wherein the at least one furin cleavage site comprises three furin cleavage sites.

8. The cleavable proinsulin construct of claim 1, wherein the proinsulin subunit comprises a C peptide, an A chain, and a B chain, and wherein the C peptide is disposed between the A chain and the B chain, wherein the at least one furin cleavage site comprises three furin cleavage sites, wherein first and second furin cleavage sites flank opposed sides of the C-peptide, and wherein a third furin cleavage site is located at a junction between the CTB subunit and the proinsulin subunit.

9. The cleavable proinsulin construct of claim 1, wherein the at least one furin cleavage site comprises an Arg Arg Lys Arg (SEQ ID NO: 3) furin cleavage sequence.

10. The cleavable proinsulin construct of claim 1, wherein the cleavable insulin construct is CTB-PFx3.

11. A DNA construct that encodes, upon expression in a plant cell, the cleavable proinsulin construct of claim 1.

12. The DNA construct of claim 11, wherein the proinsulin subunit comprises a C peptide.

13. The cleavable proinsulin construct of claim 12, wherein the proinsulin subunit further comprises an A chain and a B chain, and wherein the C peptide is disposed between the A chain and the B chain.

14. The cleavable proinsulin construct of claim 12, wherein the at least one furin cleavage comprises a cleavage site that flanks the C-peptide.

15. The cleavable proinsulin construct of claim 11, wherein the proinsulin subunit comprises a C peptide, an A chain, and a B chain, and wherein the C peptide is disposed between the A chain and the B chain, wherein the at least one furin cleavage site comprises three furin cleavage sites, wherein first and second furin cleavage sites flank opposed sides of the C-peptide, and wherein a third furin cleavage site is located at a junction between the CTB subunit and the proinsulin subunit.

16. A transgenic plant transformed with the DNA construct of claim 11.

17. The transgenic plant of claim 16, wherein the transgenic plant is selected from the group consisting of a tobacco and a lettuce plant.

18. A transgenic plant transformed with the DNA construct of claim 15.

19. The transgenic plant of claim 16, wherein the transgenic plant is selected from the group consisting of a tobacco and a lettuce plant.

20. A plant cell, wherein said plant cell comprises chloroplasts transformed to express the cleavable proinsulin construct of claim 1.

21-45. (canceled)
